(19) United States
(12) Patent Application Publication
(10) Pub. No.: US 2024/0424168 A1
(43) Pub. Date: Dec. 26, 2024

(54) SYSTEM AND METHOD FOR PREPARING AUTO GRAFT COLLAGEN FIBER SHEETS

(71) Applicant: Medos International Sarl, Le Locle (CH)

(72) Inventor: David B. Spencer, Farmington, ME (US)

(21) Appl. No.: 18/338,706

(22) Filed: Jun. 21, 2023

(51) Int. Cl. A61L 27/36; A61L 27/24; B26D 1/14

(52) U.S. Cl. CPC A61L 27/3691; A61L 27/24; A61L 27/3604; A61L 27/3662; B26D 1/14; A61L 2430/10

(57) ABSTRACT

An apparatus for making a collagen fiber sheet from a graft of any of a tendon, ligament or fascia, the graft having an outer layer of tissue surrounding a plurality of fiber bundles, each of the plurality of fiber bundles having a first diameter. The apparatus can include a surface having an opening extending therethrough, a blade coupled to the surface proximate to the opening and extending a predetermined height above the surface, and a graft retaining system configured to direct the graft toward the blade while rotating the graft about a longitudinal axis thereof during a cutting operation, wherein the graft retaining system is configured to direct the collagen fiber sheet produced by the cutting operation through the opening.

### BACKGROUND

Tendons are strong fibrous tissues that connect the muscles to bones. In the case of auto graft tendon surgery, a surgeon will typically harvest a section of healthy tendon tissue from another part of a patient's body, such as the gracilis and semi-tendinosis hamstring, patellar, or quadriceps tendon. This tissue is then prepared and surgically implanted to replace the damaged or diseased tendon, with the goal of restoring function and reducing pain in the affected area.

### SUMMARY

In one aspect, an apparatus for making a collagen fiber sheet from a graft of any of a tendon, ligament or fascia is provided. The graft can have an outer layer of flesh surrounding a plurality of fiber bundles, each of the plurality of fiber bundles having a first diameter. In an embodiment, the apparatus can include a surface having an opening extending therethrough, a blade coupled to the surface proximate to the opening and extending a predetermined height above the surface, and a tendon retaining system configured to direct the graft toward the blade while rotating the graft about a longitudinal axis thereof during a cutting operation, wherein the tendon retaining system is configured to direct the collagen fiber sheet produced by the cutting operation through the opening.

In another embodiment, the predetermined height is greater than the first diameter. In another embodiment, the first diameter is approximately 1 mm.

In another embodiment, the graft is a quadriceps tendon.

In another embodiment, the graft is generally round and has a circumference greater than or equal to 9 mm. In another embodiment, a length of the graft is greater than or equal to 7 mm.

In another embodiment, the collagen fiber sheet includes a first layer including the outer layer of flesh and at least one second layer including a layer of fiber bundles of the plurality of fiber bundles. In another embodiment, the integrity of each of the fiber bundles in the layer of fiber bundles making up the at least one second layer is preserved during the cutting operation.

In another embodiment, the tendon retaining system further includes: a pair of rollers arranged to rotate the graft about the longitudinal axis thereof, and a retaining member arranged to apply a pressure on the graft toward the surface to guide the graft through the opening guide during the cutting operation.

In another aspect, a method for making a collagen fiber sheet from a round graft is provided. In one embodiment, the graft has an outer layer of flesh surrounding a plurality of fiber bundles around a circumference of the graft. In this embodiment, the method includes: slicing the outer layer of flesh along a longitudinal axis of the graft, slicing in between the plurality of fiber bundles along a path, and opening up the graft such that a flat collagen fiber sheet is formed, the flat collagen fiber sheet having a length along the longitudinal axis and a width defined by the circumference of the graft.

In another embodiment, the flat collagen fiber sheet includes a first surface defined by the outer layer of flesh, and a second surface defined by one or more layers of adjacent fiber bundles of the plurality of fiber bundles.

In another embodiment, the integrity of the one or more layers of adjacent fiber bundles are preserved.

In another embodiment, the slicing of the outer layer of flesh and the slicing in between the plurality of fiber bundles along the path is done by a knife operated by a user.

In another embodiment, the method further includes: placing the graft on a surface of a tendon slicing apparatus, the surface including an opening extending therethrough, and a blade coupled to the surface proximate to the opening and extending a predetermined height above the surface. The method also includes directing, by a tendon retaining system of the tendon slicing apparatus, the graft toward the blade, slicing, by the blade, the outer layer of flesh along the longitudinal axis of the graft, slicing, by the blade, in between the plurality of fiber bundles along the path. The method also includes rotating, by the tendon retaining system, the graft about the longitudinal axis thereof, while slicing in between the plurality of fiber bundles along the path to produce a graft collagen fiber sheet, and directing, by the tendon retaining system, the graft collagen fiber sheet through the opening.

In another embodiment, the predetermined height of the blade is greater than a diameter of a fiber bundle of the plurality of fiber bundles. In another embodiment, the predetermined height is greater than 1 mm.

In another embodiment, the graft is a quadriceps tendon. In another embodiment, the graft is generally round and has a circumference that is greater than 9 mm.

In another embodiment, the method further includes: surgically applying the graft collagen fiber sheet to an Anterior Cruciate Ligament (ACL), a meniscus, or a Posterior Cruciate Ligament (PCL) of a human patient.

In another embodiment, the method further includes: surgically applying the graft collagen fiber sheet to a superficial surface of a rotator cuff of a human patient.

### DESCRIPTION OF DRAWINGS

These and other features will be more readily understood from the following detailed description taken in conjunction with the accompanying drawings, in which:

FIG. 1 illustrates an embodiment of a collagen fiber harvesting device as described herein;

FIG. 2 illustrates the embodiment of FIG. 1 during a collagen fiber harvesting operation as described herein;

FIG. 3 illustrates an enlarged view of a blade of the collagen fiber harvesting device of FIG. 1 during the collagen fiber harvesting operation;

FIG. 4 illustrates a graft collagen fiber sheet produced by collagen fiber harvesting operation of FIG. 2;

FIG. 5A is a top view an embodiment to a graft collagen fiber sheet produced by the collagen fiber harvesting device of FIG. 1;

FIG. 5B is a side view of the graft collagen fiber sheet of FIG. 5A, produced by the collagen fiber harvesting device of FIG. 1; and

FIGS. 6A-6D illustrate another exemplary collagen fiber harvesting operation as described herein.

It is noted that the drawings are not necessarily to scale. The drawings are intended to depict only typical aspects of the subject matter disclosed herein, and therefore should not be considered as limiting the scope of the disclosure.

### DETAILED DESCRIPTION

Traditionally, collagen fibers, including ligaments, fascia and tendons, such as the gracilis and semi-tendinosis hamstring tendons are harvested from a patient to be used to replace torn ligaments, for example, to be used in ACL reconstruction. In this use case, a remnant end of one or both of these tendons could be removed via sharp resection with a scalpel during grafting. Traditionally, the grafts are then flattened out by either scraping them out into a flat graft, or flattening them out using a mallet or the like. Tendons, such as the gracilis and semi-tendinosis hamstring tendons and others, however, are not monolithic, but rather, have discrete axial tendon bundles that are surrounded by a resilient skin. Accordingly, under the traditional flattening methods described above, the integrity of the bundles is not preserved, which can have adverse effects on the strength of the flattened graft.

The apparatus and method described herein can address the aforementioned shortcomings. For example, one or more embodiments of the apparatus and methods herein can provide a graft collagen fiber sheet wherein the integrity of the discrete axial collagen fiber bundles are preserved by carefully cutting into the skin of the collagen fiber and opening up the collagen fiber into a flat sheet. In some embodiments, the apparatus described herein can include a surface having an opening extending therethrough, a blade coupled to the surface proximate to the opening and extending a predetermined height above the surface, and a collagen fiber retaining system arranged to direct the graft toward the blade during a collagen fiber sheet preparing operation. The resulting graft collagen fiber sheet can include a first surface made up of the resilient outer skin of the collagen fiber, and a second surface made up of at least one layer of parallel axial tendon bundles.

Though the embodiments described herein often refer to collagen fiber harvesting and therefore creating a collagen fiber graft, the apparatuses and methods described herein can be used with tissue structures, including ligaments, fascia, or tendons. In some embodiments, the apparatuses and methods described herein may be used with non-orthopaedic structures, for example, the pericardium or pleura.

The apparatus and method described herein advantageously provide a sheet of graft that can be used in a variety of surgical operations, wherein the integrity of the discrete axial collagen fiber bundles within the tendon is preserved.

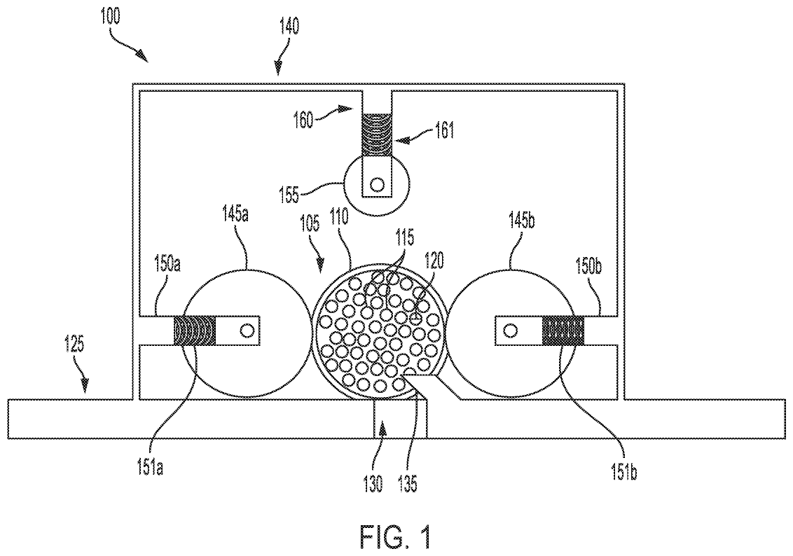

FIG. 1 illustrates an embodiment of an apparatus 100 for making a collagen fiber sheet from a graft 105 that has an outer layer of flesh 110 surrounding a plurality of fiber bundles 115. In some cases, the outer layer of flesh 110 can have a first thickness and the plurality of fiber bundles 115 can have a first diameter 120. For example, in some embodiments, the graft 105 can be a graft quadriceps tendon. In some embodiments, the graft 105 can be generally round, as illustrated, and can have a circumference that is greater than or equal to 9 mm. In some embodiments, the graft 105 can be greater than or equal to 7 mm in length. In some embodiments, the apparatus 100 can include a surface 125 including an opening 130 extending therethrough. The apparatus 100 can also include a blade 135 coupled to the surface 125, proximate to the opening 130 and extending a predetermined height above the surface 125, as discussed in greater detail below. In some embodiments, the blade 135 can be removably coupled to the surface 125 to allow for replacement of the blade, however, in some embodiments, the blade 135 can be formed integrally with the surface 125. The apparatus 100 can further include a collagen fiber retaining system 140 that can be configured to direct the graft 105 toward the blade 135 while rotating the graft 105 about a longitudinal axis thereof during a cutting operation. In some embodiments, the collagen fiber retaining system 140 can include a longitudinal retaining mechanism including rollers 145a, 145b, supports 150a, 150b, and corresponding spring members 151a, 151b. In some embodiments, the rollers 145a, 145b can have a rough or higher friction surface finish that allows then to maintain a grip on the graft 105 during a cutting operation, as will be discussed in greater detail below. The collagen fiber retaining system 140 can also include an axial retaining mechanism including roller 155, support 160, and corresponding spring member 161. The axial retaining mechanism, by way of the spring member 161 can be configured to apply a downward pressure on the graft 105 during a cutting operation, as discussed in greater detail below.

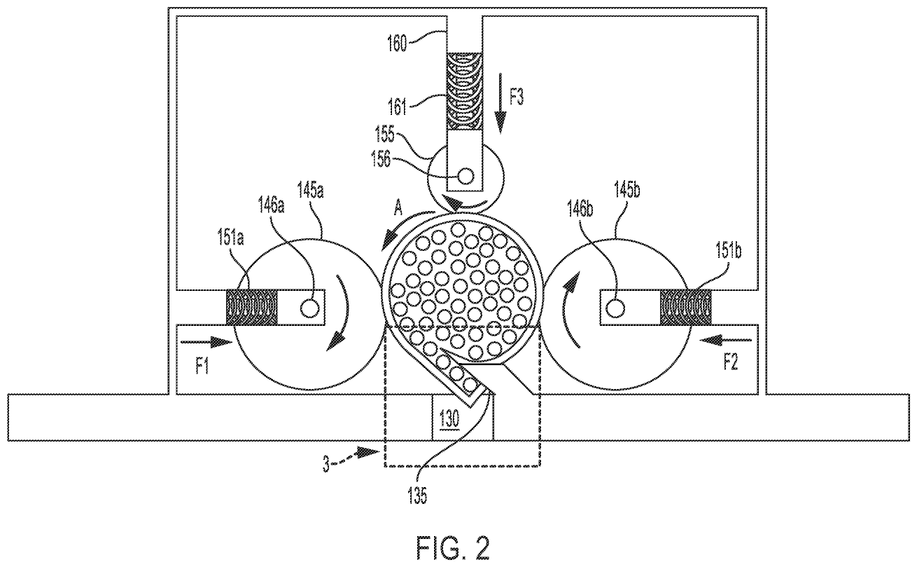

FIG. 2 illustrates the apparatus 100 during a cutting operation. The cutting operation, which uses apparatus 100, is described below with reference to FIGS. 1-2. In some embodiments, the graft 105 can be placed on the surface 125 in between the longitudinal rollers 145a. 145b. In some embodiments, spring elements 151a, 151b can provide longitudinal forces F1, F2 respectively, on the graft 105, and the longitudinal rollers 145a, 145b can be configured to rotate about their respective central axes 146a, 146b to rotate the graft in a direction A. Additionally, the axial spring member 161 can be configured to apply a downward force F3 on the graft 105, and the roller 155 can be configured to rotate about its central axis 156. Forces F1. F2, and F3 can be configured to longitudinally and axially direct the graft 105 toward the blade 135. As the rollers 145a, 145b and 155 rotate the graft 105 about direction A, the blade 135 can be configured to cut into the graft 105, as discussed in greater detail below.

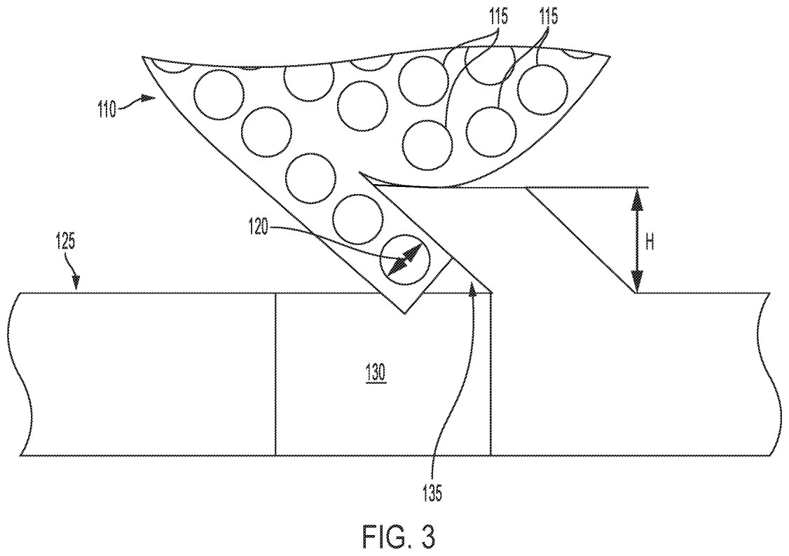

FIG. 3 illustrates an enlarged view of the section 3 of FIG. 2 showing the blade 135 cutting into the graft 105 during a collagen fiber harvesting operation to make a graft collagen fiber sheet. As illustrated in FIG. 3, the blade 135 can be configured to extend a predetermined height H above the surface 125. In some embodiments, the height H can be greater than the first diameter 120 of the plurality of fiber bundles 115 so that the integrity of the plurality of fiber bundles 115 can be maintained when preparing the graft collagen fiber sheet. For example, in some embodiments, the first diameter 120 can be about 1 mm. Accordingly, in some embodiments, the height H can be greater than 1 mm.

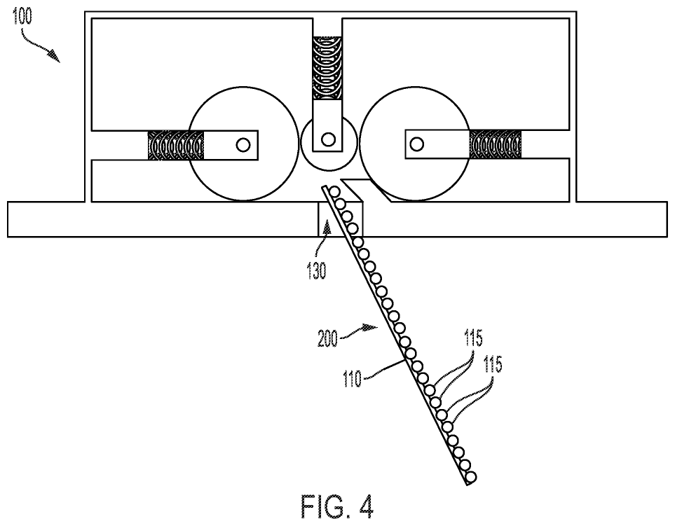

FIG. 4 illustrates the apparatus 100 at the end of the cutting operation, whereby a graft collagen fiber sheet 200 is produced out of the graft 105 and fed out of the opening 130 of the apparatus 100. In some embodiments, the graft collagen fiber sheet 200 can include a first layer made up of the outer layer of tissue 110, and at least one second layer made up of the plurality fiber bundles 115. As mentioned above, the integrity of each of the fiber bundles 115 in the layer of fiber bundles making up the at least one second layer can preserved during the cutting operation.

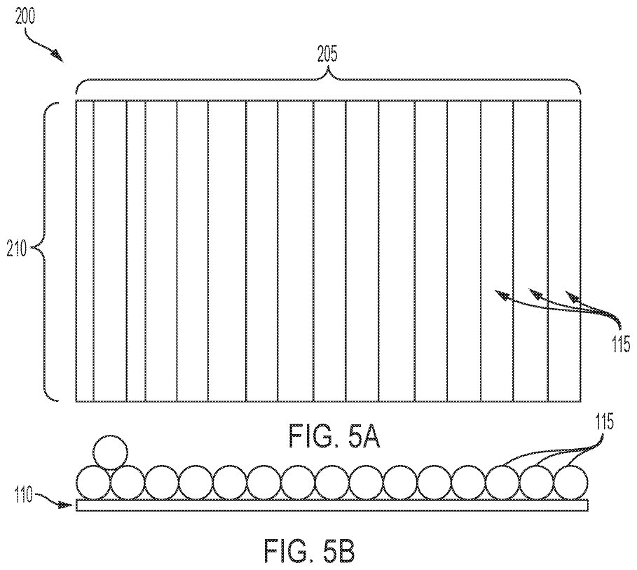

FIG. 5A illustrates a top view of the graft collagen fiber sheet 200 that is produced by the apparatus 100 using the graft 105. As illustrated in FIG. 5, the graft collagen fiber sheet 200 can have a width 205 and a length 210. In some embodiments, the graft collagen fiber sheet 200 can have a width 205 that is equal to the circumference of the graft 105 of FIG. 1. Accordingly, in some embodiments, the collagen fiber sheet 200 can have a width 205 that is greater than or equal to 9 mm. Similarly, the graft collagen fiber sheet 200 can have a length 210 that is equal to the length of the graft 105 of FIG. 1. Accordingly, in some embodiments, the graft collagen fiber sheet 200 can have a length 210 that is greater than or equal to 7 mm. FIG. 5B illustrates a side view of the graft collagen fiber sheet 200 of FIG. 5A.

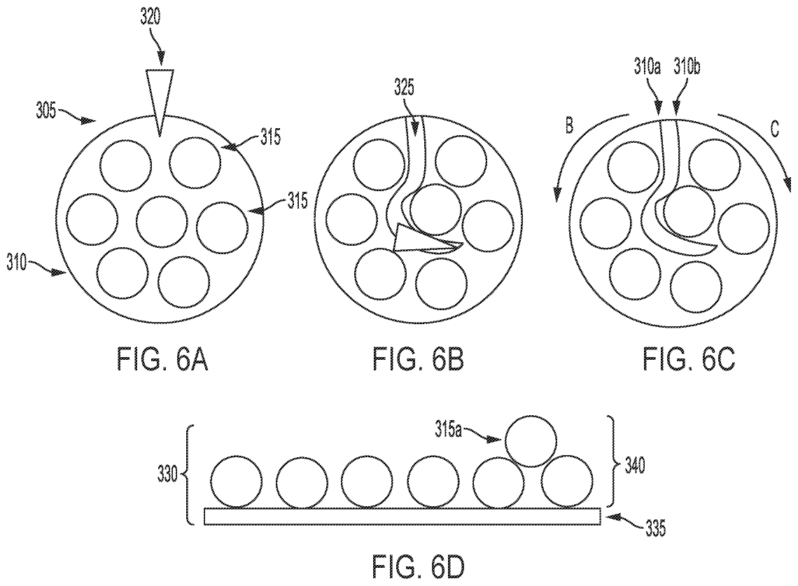

FIGS. 6A-6D illustrate another exemplary collagen fiber harvesting operation 300 for preparing a graft collagen fiber sheet 330 out of a graft 305. In some cases, the graft 305 can be similar to graft 105 of FIG. 1. As illustrated in FIG. 6A, during the collagen fiber harvesting operation 300, a user can cut into an outer layer of tissue 310 of the graft 105, along a longitudinal axis thereof, using a blade 320. In some embodiments, the blade 320 can be part of a surgical knife or the like. The outer layer of tissue 310 can surround a plurality of fiber bundles 315 that can extend along a length of the tendon 305. As illustrated in FIG. 6B, the user can navigate the blade 320 along a path 325 in order to slice in between the plurality of fiber bundles 315. By slicing the graft 305 along the path 325, the integrity of the fiber bundles 315 can be maintained, which can advantageously increase the overall strength of the graft collagen fiber sheet 330 that is produced. As illustrated in FIG. 6C-6D, the user can then flatten the graft 305 into the graft collagen fiber sheet 330 by opening a first end 310a of the outer layer of tissue 310 along a direction B and opening a second end 310b of the outer layer of tissue 310 along a direction C. In some embodiments, the graft collagen fiber sheet 330 can includes a first surface 335 defined by the outer layer of tissue 310, and a second surface 340 defined by one or more layers of adjacent fiber bundles 315. In some embodiments, the graft collagen fiber sheet 330 is not perfectly symmetrical, but can rather include one or more askew fiber bundles 315a across the second surface 340. In some embodiments, an additional step may be performed to remove at least one askew fiber bundle (e.g., fiber bundle 315a) from the graft collagen fiber sheet 330. Similar to the graft collagen fiber sheet 200 of FIG. 5, in some embodiments, the graft collagen fiber sheet 330 can have a width that is approximately equal to the circumference of the graft 305. In some embodiments, the collagen fiber sheet 330 can have a width that is greater than or equal to 9 mm. In some embodiments, the graft collagen fiber sheet 330 can have a length that is equal to the length of the graft 305. In some embodiments, the graft collagen fiber sheet 330 can have a length that is greater than or equal to 7 mm.

In some embodiments, the graft collagen fiber sheet 200 of FIG. 5 or the graft collagen fiber sheet 330 of FIG. 6D can be surgically applied to any of an Anterior Cruciate Ligament (ACL), a meniscus, and a Posterior Cruciate Ligament (PCL) of a human patient. Alternatively, in some embodiments, the graft collagen fiber sheet 200 of FIG. 5 or the graft collagen fiber sheet 330 of FIG. 6D can be surgically applied to a superficial surface or a deep surface of a rotator cuff of a human patient. Further, in some embodiments, the graft collagen fiber sheet 200 of FIG. 5 or the graft collagen fiber sheet 330 of FIG. 6D can be used to strengthen/bulk a hip labrum or a shoulder labrum of a human patient.

Certain exemplary embodiments have been described to provide an overall understanding of the principles of the structure, function, manufacture, and use of the systems, devices, and methods disclosed herein. One or more examples of these embodiments have been illustrated in the accompanying drawings. Those skilled in the art will understand that the systems, devices, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention. Further, in the present disclosure, like-named components of the embodiments generally have similar features, and thus within a particular embodiment each feature of each like-named component is not necessarily fully elaborated upon.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

One skilled in the art will appreciate further features and advantages of the invention based on the above-described embodiments. Accordingly, the present application is not to be limited by what has been particularly shown and described, except as indicated by the appended claims. All publications and references cited herein are expressly incorporated by reference in their entirety.

1. An apparatus for making a collagen fiber sheet from a graft of any of a tendon, ligament or fascia, the graft having an outer layer of tissue surrounding a plurality of fiber bundles, each of the plurality of fiber bundles having a first diameter, the apparatus comprising:
a surface having an opening extending therethrough;
a blade coupled to the surface proximate to the opening and extending a predetermined height above the surface; and
a graft retaining system configured to direct the graft toward the blade while rotating the graft about a longitudinal axis thereof during a cutting operation, wherein the graft retaining system is configured to direct the collagen fiber sheet produced by the cutting operation through the opening.

2. The apparatus of claim 1, wherein the predetermined height is greater than the first diameter.

3. The apparatus of claim 1, wherein the first diameter is approximately 1 mm.

4. The apparatus of claim 1, wherein the graft is a quadriceps tendon.

5. The apparatus of claim 4, wherein the graft is generally round and has a circumference greater than or equal to 9 mm.

6. The apparatus of claim 1, wherein the predetermined height is configured to be adjustable curing the cutting operation.

7. The apparatus of claim 1, wherein the collagen fiber sheet includes a first layer comprising the outer layer of flesh and at least one second layer comprising a layer of fiber bundles of the plurality of fiber bundles.

8. The apparatus of claim 7, wherein the integrity of each of the fiber bundles in the layer of fiber bundles making up the at least one second layer is preserved during the cutting operation.

9. The apparatus of claim 1, wherein the tendon retaining system further comprises:
a pair of rollers configured to rotate the graft about the longitudinal axis thereof; and
a retaining member configured to apply a pressure on the graft toward the surface to guide the graft through the opening guide during the cutting operation.

10. A method for making a collagen sheet from a round graft of any of a tendon, ligament or fascia, the graft having an outer layer of tissue surrounding a plurality of fiber bundles around a circumference of the graft, the method comprising:
slicing the outer layer of tissue along a longitudinal axis of the graft;
slicing in between the plurality of fiber bundles along a path; and
opening up the graft such that a flat collagen sheet is formed, the flat tendon sheet having a length along the longitudinal axis and a width defined by the circumference of the graft.

11. The method of claim 10, wherein the flat collagen sheet includes a first surface defined by the outer layer of tissue, and a second surface defined by one or more layers of adjacent fiber bundles of the plurality of fiber bundles.

12. The method of claim 10, wherein the integrity of the one or more layers of adjacent fiber bundles are preserved.

13. The method of claim 10, wherein the slicing of the outer layer of tissue and the slicing in between the plurality of fiber bundles along the path is done by a knife operated by a user.

14. The method of claim 10, wherein the graft is a quadriceps tendon.

15. The method of claim 10, further comprising:
surgically applying the graft sheet to an Anterior Cruciate Ligament (ACL), a meniscus, or a Posterior Cruciate Ligament (PCL) of a human patient.

16. The method of claim 10, further comprising:
surgically applying the collagen sheet to a superficial surface or a deep surface of a rotator cuff of a human patient.

17. A method comprising:
placing a graft of any of a tendon, ligament or fascia on a surface of a graft slicing apparatus;
directing the graft toward a blade coupled to the surface of the graft slicing apparatus and extending a predetermined height above the surface;
slicing, by the blade, the outer layer of tissue along the longitudinal axis of the graft;
slicing, by the blade, in between the plurality of fiber bundles along a path;
rotating the graft about the longitudinal axis thereof, while slicing in between the plurality of fiber bundles along the path to produce a graft collagen sheet; and
directing the graft collagen sheet through an opening extending through the surface proximate to the blade.

18. The method of claim 17, wherein the predetermined height of the blade is greater than a diameter of a fiber bundle of the plurality of fiber bundles.

19. The method of claim 17, wherein the predetermined height is greater than 1 mm.

20. The method of claim 17, wherein the graft is generally round and has a circumference that is greater than 9 mm.
